(19) United States
(12) Patent Application Publication
(10) Pub. No.: US 2010/0305676 A1
(43) Pub. Date: Dec. 2, 2010

(54) COCHLEAR IMPLANT ELECTRODE ASSEMBLY

(76) Inventors: Fysh Dadd, Lane Cove, NSW (AU); Claudiu Treaba, Centennial, CO (US); C. Roger Leigh, North Epping (AU)

Correspondence Address: JOHN S. PRATT, ESQ. (Cochlear);KILPATRICK STOCKTON LLP, 1100 PEACHTREE STREET, SUITE 2800, ATLANTA, GA, 30309, US

(21) Appl. No.: 12/743,368

(22) PCT Filed: Nov. 17, 2008
(86) PCT No.: PCT/AU08/01712

§ 371 Date: Aug. 18, 2010

(30) Foreign Application Priority Data
Nov. 16, 2007 (AU) 2007906283

(51) Int. Cl. A61F 11/04; A61N 1/05

(52) U.S. Cl. 607/137

(57) ABSTRACT

Disclosed is a lead for a cochlear implant modified so as to provide increased robustness to the lead. In one aspect, the lead has a helix region having an increased length such that it extends into a mastoid cavity of a patient when in situ. Also disclosed is a cochlear implant having a lead as described herein. In another aspect, the lead has an increased lead angle between the helix region and the transition region.

### CROSS-REFERENCE TO RELATED APPLICATION

The present invention is a National Stage Application of International Application No. PCT/AU2008/001712, filed Nov. 17, 2008, which claims priority from Australian Patent Application No. 2007906283, filed Nov. 16, 2007, which is hereby incorporated herein by reference herein.

### BACKGROUND

1. Field of the Invention

The present invention relates generally to cochlear implants, and more particularly, to an electrode assembly of a cochlear implant.

2. Related Art

Medical implants are used in many areas of medicine to enhance the length and/or quality of the life of the implant recipient. Such implants include pacemakers, controlled drug delivery implants and cochlear implants.

A cochlear implant allows for electrical stimulating signals to be applied directly to the auditory nerve fibers of the patient, allowing the brain to perceive a hearing sensation approximating the natural hearing sensation. These stimulating signals are applied by electrode contacts of an electrode array implanted in the patient's cochlea.

The electrode array is connected to a stimulator unit (by way of a lead) which generates the electrical signals for delivery to the electrode array. The stimulator unit in turn is operationally connected to a signal processing unit which also contains a microphone for receiving audio signals from the environment, and for processing these signals to generate control signals for the stimulator.

The signal processing unit is in practice, located externally to the patient and the stimulator is implanted within the patient, usually near the mastoid on the patient's skull and underneath the surrounding tissue. The processor and stimulator may communicate by various wireless means including by a radio frequency link.

FIG. 1 shows a typical structure of a cochlear implant 100 defining the different regions. Shown there is the stimulator 10, to which is connected electrode assembly or lead 20. Electrode assembly 20 includes helix region 21, transition region 22, proximal region 23 and intra-cochlear region 24. Intra-cochlear region 24 includes the electrode contacts, which in use are inserted into the patient's cochlea. Proximal region 23 and intra-cochlear region 24 together form the electrode array.

The stimulator 10 generates the electrical signals that are applied to the patient's auditory nerves in the cochlea via the lead and through the electrode wires (not shown) and respective electrode contacts of the electrode array.

The helix region 21 contains the electrode wires of the lead (for example 22 wires). These wires are wound in a helical arrangement in this region.

The transition region is the region between the helix and the proximal region. This is the region proximal to the facial recess/posterior tympanotomy when the electrode assembly is implanted in a patient's cochlea.

The proximal region 23 is, when in situ, outside of the cochlea (proximal to cochleostomy). It is defined as the region between the ribs of the electrode array (which are at the cochleostomy) and the stylet exit. The stylet exit is just outside of the posterior tympanotomy.

The intra-cochlear region 24 is the active portion of the electrode array that contains the electrode contacts. The whole of this portion is intra-cochlear, i.e. apical to the cochleostomy.

A typical length of the helix region 21 is about 45 mm, and a typical length of the transition region 22 is about 24 mm.

FIG. 2 shows the implant 100 implanted into a patient. Shown there is the stimulator 10 lying in a bed 51 formed in the skull 50 of a patient with a skin flap 51a of the scalp of the patient's head folded back. The bed 51 provides a space for location of the stimulator 10 to retain it in place in the patient's skull and to minimize protrusion of the stimulator package from the skull when in place. A gutter 52 is also provided to accommodate the lead 20 coming from stimulator 10. A hole is drilled into the mastoid bone to allow the lead 20 to enter the middle ear and provide access to the round window of the cochlea 55. The area of bone that is removed to provide access to the cochlea 55 is referred to as the mastoid cavity 53. FIG. 2 shows that the lead 20 is provided long enough to assist the surgeon in manipulating the lead 20 into the cochlea, as well as to take account for any growth in the patient's skull, if implanted at a young age. Accordingly, the surgeon typically forms a loop in the transition region 22 of the lead 20 that is then placed in the mastoid cavity 53, to account for any excess lead length.

The wires connecting the electrodes to the stimulator are very thin and can be easily damaged during manufacture of the lead, transport, storage, surgical insertion or even when in the patient, through either external impact to the patient's head or simply by movement of the lead due to growth of the patient's skull or diurnal activities, such as chewing.

Damage to the wires can eventually result in the wires or insular coating of the wires breaking due to fatigue, which typically cause faults such as open or short circuits to develop in or between the wires of the electrode assembly. These faults can cause insufficient stimulation at some nerves and overstimulation at others. Furthermore, the damage that causes faults to develop in the lead is difficult to detect and may only be evident after the lead is implanted due to deteriorating performance of the implant. As a consequence, frequent remapping of a cochlear implant is often required in order to correct, or at least mitigate, the progressive malfunction or loss of electrode contacts caused by these faults.

### SUMMARY

In accordance with one embodiment of the present invention, a cochlear implant electrode assembly for operably connecting to an implantable stimulator unit and having electrode contacts disposed at the distal end thereof for implantation into a cochlea of a patient, the electrode assembly is disclosed. The cochlear implant comprising: a helix region, adjacent the stimulator unit, containing wires connecting the stimulator unit to the electrode contacts, wherein the wires are wound in a helical arrangement; an electrode array comprising an intra-cochlear region containing the electrode contacts and configured to be completely implanted in the cochlea, and a proximal region adjacent the intra-cochlear region and configured to be implanted extra cochlear; and a transition region contiguous with and disposed between the helix and proximal regions, wherein the length of the transition region has a length of about 5 mm.

In accordance with a second embodiment of the present invention a cochlear implant is disclosed. The cochlear implant comprising: an implantable stimulator unit; and an electrode assembly operably connected to the stimulator unit and having electrode contacts disposed at the distal end thereof for implantation into a cochlea of a patient, the electrode assembly comprising: a helix region, adjacent the stimulator unit, containing wires connecting the stimulator unit to the electrode contacts, wherein the wires are wound in a helical arrangement; an electrode array comprising an intra-cochlear region containing the electrode contacts and configured to be completely implanted in the cochlea, and a proximal region adjacent the intra-cochlear region and configured to be implanted extra cochlear; and a transition region contiguous with and disposed between the helix and proximal regions, wherein the length of the transition region has a length of about 5 mm.

In accordance with a third embodiment of the present invention a cochlear implant electrode assembly for operably connecting to an implantable stimulator unit and having electrode contacts disposed at the distal end thereof for implantation into a cochlea of a patient, the electrode assembly is disclosed. The cochlear implant electrode assembly comprising: a helix region, adjacent the stimulator unit, containing wires connecting the stimulator unit to the electrode contacts, wherein the wires are wound in a helical arrangement; and an electrode array contiguous with and adjacent to the helix region, the electrode array comprising an intra-cochlear region containing the electrode contacts and configured to be completely implanted in the cochlea, and a proximal region adjacent the intra-cochlear region and configured to be implanted extra cochlear.

### BRIEF DESCRIPTION OF THE DRAWINGS

Various aspects and embodiments of the present invention will now be described in detail with reference to the following drawings in which:

FIG. 1 is a side view of a conventional electrode assembly or lead for a cochlear implant;

FIG. 2 is a side view of a cochlear implant with the conventional lead of FIG. 1 being implanted in a patient;

FIG. 3 is a side view of a cochlear implant electrode assembly according to one aspect of the present invention;

FIG. 4 is a side view of a portion of the electrode assembly illustrated in FIG. 3;

FIG. 5 is a side view of the cochlear implant electrode assembly of FIGS. 3 and 4 implanted in a patient;

FIGS. 6A is a side view of a conventional electrode assembly or lead depicting a lead angle of 3.5 degrees;

FIG. 6B is a side view of a portion of an embodiment of an electrode assembly of the present invention depicting a lead angle of 10 degrees;

FIG. 6C is a side view of a portion of an embodiment of an electrode assembly of the present invention with an increased lead angle and tapered transition region; and

FIG. 7 is a side view of an alternative embodiment of an electrode assembly of the present invention.

### DETAILED DESCRIPTION

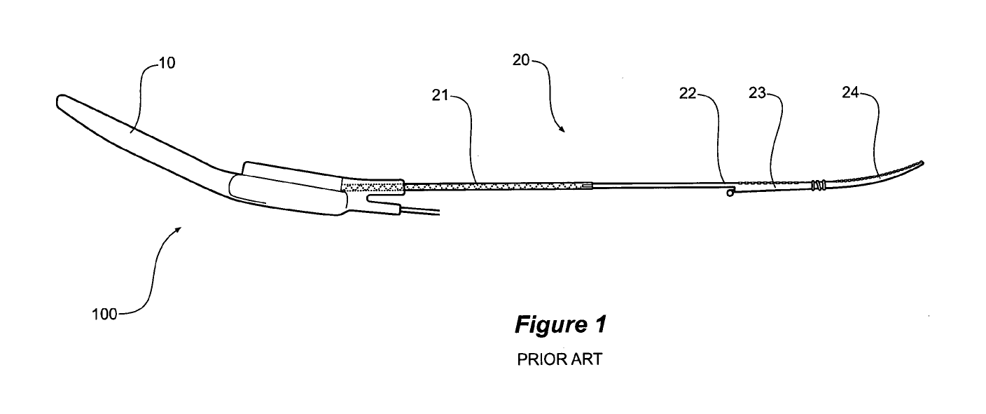

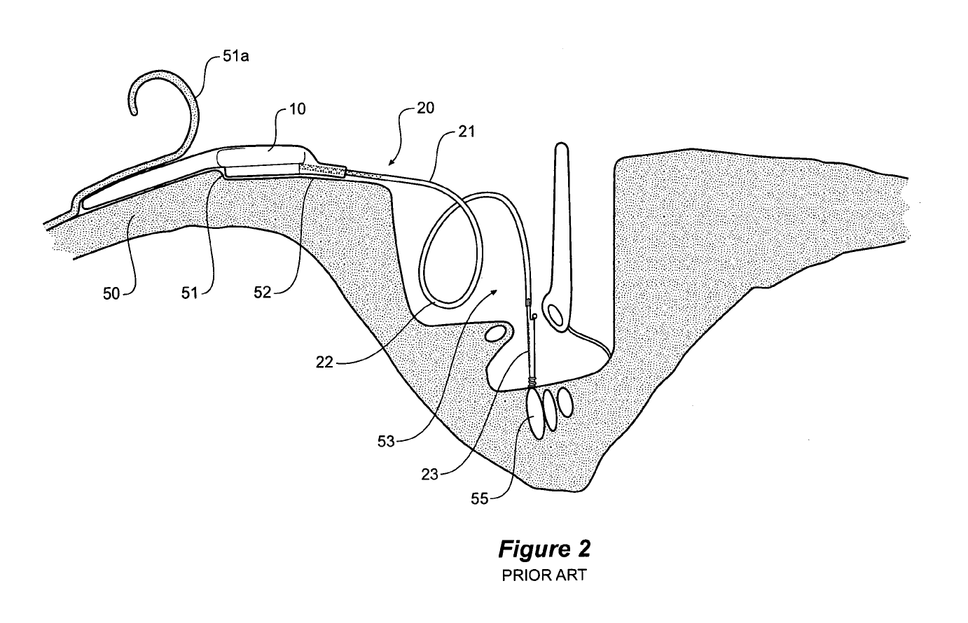

It has been discovered that the conventional electrode assembly or lead 20 of FIG. 1 is vulnerable to damage at the transition region 22 due to its location on the lead 20. As shown in FIG. 2, the transition region 22 is located on the conventional lead 20 to lie outside the mastoid cavity 53 when implanted in a patient such that it is exposed to external impact or movement. According to one aspect of the present invention, the lead 20 has been redesigned so that the interface between the helix region 21 and the transition region 22 lies within the mastoid cavity 53 when the implant is in situ, the helix region 21 being the only part of the lead 20 that lies outside the mastoid cavity 53. This reduces the risk of damage to the wires of the lead from external impact to the patient's skull post implantation, and/or damage from other activities such as chewing due to the ability of the helix region to distribute force evenly along its length and elastically stretch without the wires breaking.

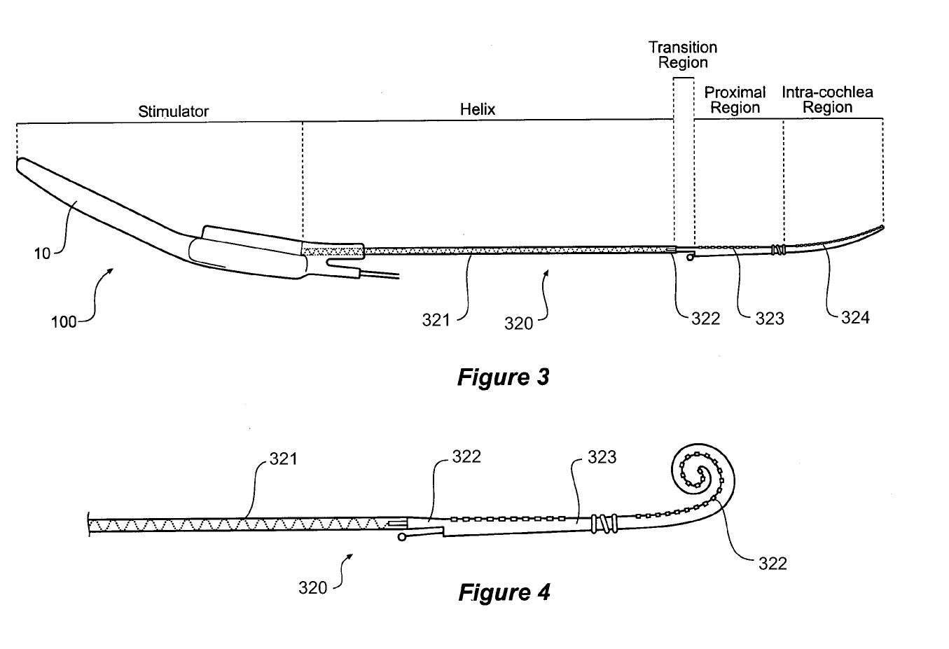

FIG. 3 shows a lead 320 according to one aspect of the present invention. In FIG. 3, it can be seen that although the lead 320 is the same length as the lead 20 of FIG. 1, the length of the helix region 321 is greater that the length of the helix region 21 of FIG. 1, and conversely, the length of the transition region 322 is smaller than the transition region 22 of the electrode assembly shown in FIG. 1.

In one form, the length of the helix region 321 is greater than about 1000% of the length of the transition region 322. In one form, the length of the helix region 321 is between about 1000% and about 1300% of the length of the transition region 322. In one form, the length of the helix region 321 is about 63 mm and the length of the transition region 322 is about 5 mm. However, one of ordinary skill in the art will appreciate that the length of the helix region 321 allows it to extend into the mastoid cavity 53 in order to provide greater protection to the electrode assembly and will depend on the distance between the implantation site selected for implantation of the stimulator 10 and the mastoid cavity 53. Accordingly, the length of the helix region 322 may range anywhere from 5 mm to 250 mm.

FIG. 4 shows a close-up view of electrode assembly 320 as shown in FIG. 3 according to this aspect of the present invention. As can be seen, the transition region 322 has been greatly shortened to about 5 mm in this example. As can also be seen, the transition region 322 has also been designed to taper from the cross-sectional dimensions of the helix region 321 to the cross-sectional dimensions of the proximal region 323. This tapering provides increased robustness over prior art leads as there is no “step” or discontinuity that would otherwise result in a stress concentrator between the helix and transition regions.

Another aspect of the new design provides for a transition region 322 with a greater minimum cross-sectional area than in previous designs. For example the diameter of the transition region 322 in FIG. 4 tapers from about 1.2 mm at the interface with the helix region 321 down to about 0.8 mm at the interface with the proximal region 323. This provides a minimum cross-sectional area of about 0.8×0.9 mm. A greater diameter or thickness contributes to increased robustness.

In one example, the silicone chosen for the transition region 22 is Nusil Med 4860, available from suppliers such as Nusil or Dow Corning. This has a Shore A hardness of 60. In another example, the silicone chosen for the transition region is Dow Corning Silastic 7-4860, biomedical grade LSR.

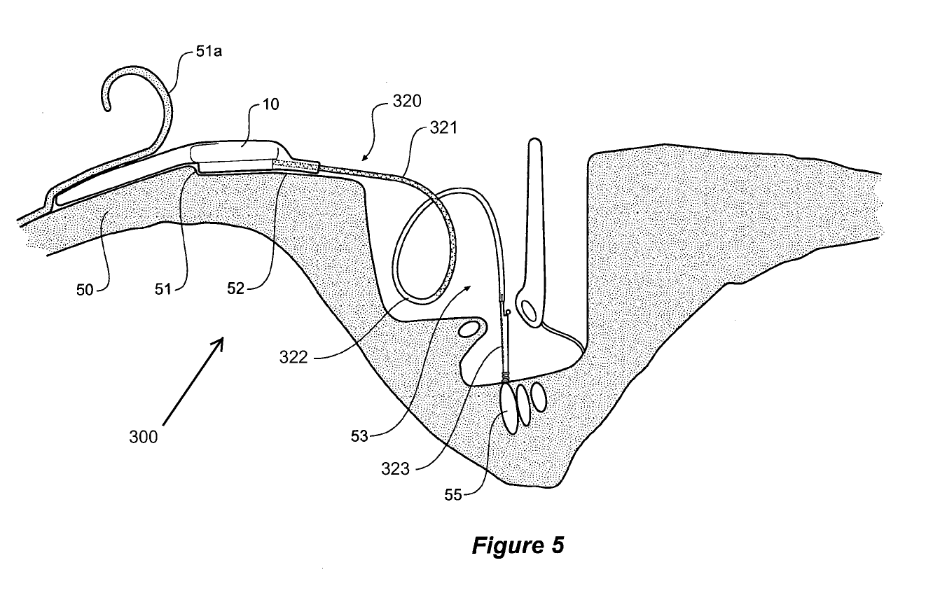

FIG. 5 shows the implant 300 having a lead 320 designed according to the various aspects of the present invention, being implanted into a patient. Shown there is the stimulator 10 lying in a bed 51 formed in the skull 50 of a patient with a skin flap 51a of the scalp of the patient's head folded back. The bed 51 provides a space for location of the stimulator 10 to retain it in place in the patient's skull and to minimize protrusion of the stimulator package from the skull when in place. A gutter 52 is also provided to accommodate the lead 320 coming from stimulator 10. A hole is also formed in the mastoid bone to allow the lead 320 to enter the mastoid cavity 53 for access to the round window of the cochlea 55.

In this arrangement, it can be seen that the helix region 321 extends into the mastoid cavity 53, such that the interface between the helix region 321 and transition region 322 is protected from external impact or other stresses that may contribute to breakage of wires.

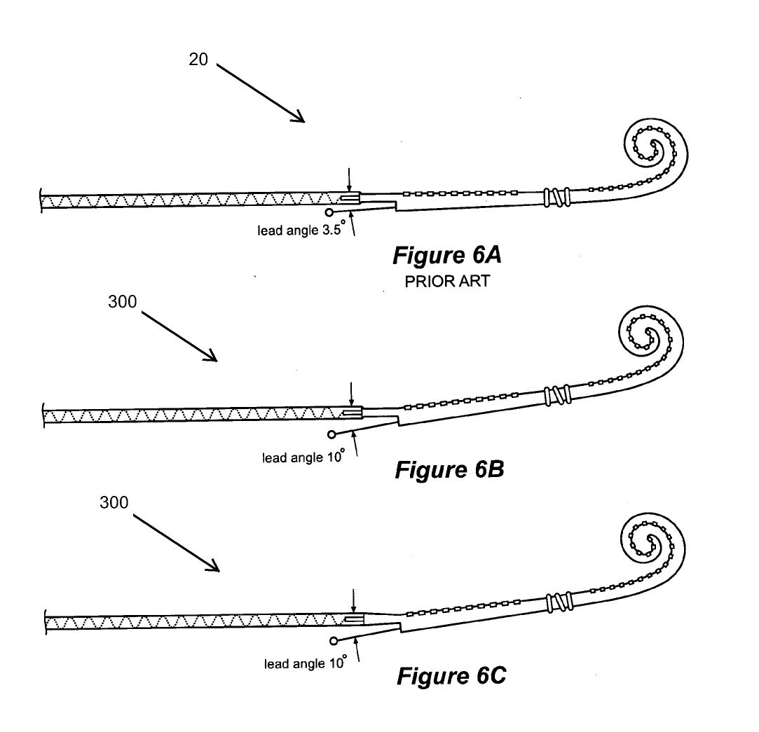

According to a further aspect of the present invention, the new design provides for a greater lead angle between the transition region 322 and the proximal region 323 as can also be seen in FIGS. 6A and 6B. FIG. 6A shows a conventional lead 20 for comparison with the embodiment 300 of the present invention shown in FIG. 6B. The lead angle has increased to 10° in the embodiment shown in FIG. 6B, from the conventional design 20 having a lead angle of 3.5° as shown in FIG. 6A.

The greater lead angle allows the helix region 321 to be located within the mastoid cavity 53, without having the larger outer diameter of the helix region 321 negatively affecting access to the stylet and or surgical visibility during insertion of the intra-cochlear region 324 into the cochlea 55.

It will be understood that the increased lead angle can take on any desired value, including 4°, 4.5°, 5°, 6°, 7°, 8°, 9°, 10°, 11°, 12°, 13°, 14°, 15° etc. Alternatively, the “angle” could be replaced by a curve.

FIG. 6C shows the option of combining the feature of the increased lead angle as shown in FIG. 6B with the feature of the tapered transition region as shown in FIG. 4.

The following description provides instructions as to how to manufacture one example of a lead according to an aspect of the present invention.

Weld the electrode contacts as known, for example, in U.S. Pat. No. 6,421,569. In one embodiment, this includes the following steps:

a) The 22 contacts are formed by slicing 0.3 mm wide sections of Platinum Tube;

b) The contacts are placed in a Welding Jig and squashed to a U shape;

c) A bundle of 22 wires is placed in the Welding Jig;

d) Each wire is connected to a contact (e.g., by welding). (The strand travels from the contact proximally in bottom of all the proximal U-shaped contacts.)

A Welded Sub-assembly is then formed (as known, for example in U.S. Pat. No. 6,421,569.) In one embodiment, this includes the following steps:

a) A droplet of silicone is then placed in the trough of each electrode contact to secure the wires.

b) The production stylet (PTFE coated wire) is pressed on top of the strands and silicone in the troughs of the electrode contacts (this stylet is removed later and forms the lumen).

c) Each electrode trough is then partially filled with more silicone.

d) The sub-assembly is then placed in an oven to cure the silicone.

e) The assembly is then removed from the straight die.

The electrode array is then molded (as known, for example, in U.S. Pat. No. 6,421,569.) In one embodiment, this includes the following steps:

a) The sub-assembly is carefully curved to match the shape of a curved molding die. The assembly is then placed in the curved molding die with the contacts being located closer to the medial side (inside of the curve).

b) The space in the die is packed with silicone material.

c) A matching die cover is placed over the assembly and pressed down.

d) The die is then placed in an oven to cure the silicone.

e) The die is then open to allow the resulting electrode array to be removed from the die.

Manufacturing a transition region by overmolding. In one embodiment, this includes the steps of:

a) The wires in the transition region are coated with a thin layer of RTV (silicone adhesive);

b) The thin coating is placed in the oven and the silicone cured.

c) The wire bundle is then placed in a transition region molding die.

d) Silicone is injected into the molding die.

e) The molding die is placed in the oven and the silicone is cured.

A helix is then formed, as known in the art. In one embodiment, this includes the following steps:

a) The wires in the helix region are coated in a thin layer of silicone.

b) The thin coating is placed in the oven and the silicone cured.

c) The wires are wound around a mandrel.

d) The mandrel is removed.

e) A silicone tube is threaded over the helixed wires.

f) The tube is injected with silicone.

g) The silicone is cured in an oven.

Forming the new transition region 322 using the above described overmolding technique, as compared to making the transition region 322 using the conventional technique of threading the wires through a silicone tube and then backfilling the tube with silicone compound (known in the prior art as “tube injection” molding), creates better quality silicone curing and thus more robust silicone to protect the wires from damage that may occur during handling, transport, and insertion of the cochlear implant 100.

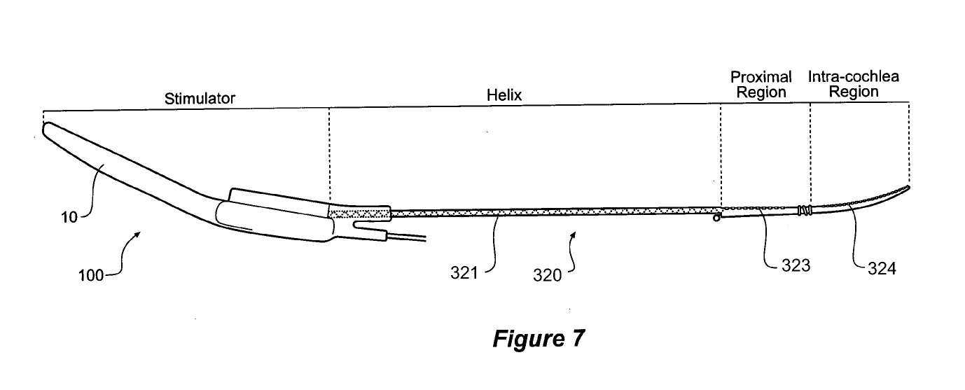

As shown in FIG. 7, in another form of the present invention, the lead 320 is formed with no transition region at all, with the helix region 321 interfacing directly with the proximal region 323. However, it should be appreciated that the proximal region 323 can be omitted from the design of the lead 320 instead of the transition region, such that transition region 322 as shown in FIG. 3, extends further along the lead 320 to interface directly with the intra-cochlear region 324.

While various embodiments of the lead 320 have been discussed comprising at least a transition region 322 and/or a proximal region 323 between the helix region 321 and the intra-cochlear region 324, it will be appreciated that the lead 320 can also be formed without a transition region 322 and a proximal region 323, such that the helix region 321 interfaces directly with the intra-cochlear region 324.

In yet a further modification of the invention as shown in FIG. 3, the lead 20 can include undulating wires (not shown) in the transition region 21 to provide a strain relief component to the transition region 21.

Disclosed herein is a lead design having a number of different features, which together, or in isolation, provide significant advantages over prior art leads. These advantages include:

A more robust electrode, resulting in higher manufacturing yield rates (due to less damage to the lead during manufacturing; a lower failure rate during surgery; a lower failure rate in situ; a more flexible transition region which is beneficial during surgery (facilitating manipulation of the array by the surgeon during surgery).

It will be understood that while the various aspects of the present invention have been described in relation to a specific embodiment, many variations and modifications may be made within the scope of the appended claims. For example, while the following features:

Shorter transition region (any size down to zero, i.e. no transition region)

Larger diameter transition region

Tapered Transition region

Larger lead angle

Harder silicone

Longer Helix (could lengthen, completely into the proximal region)

Overmoulding transition region

have been described in combination, it will be understood that each feature could be taken on its own to provide an improved lead design, or two or more of the features could be combined to provide an improved lead design.

For example a lead 20 could be provided having a tapering transition region from the helix region to proximal region, with no change in length of the helix or transition region; a thicker diameter transition region could be provided with no other dimensional changes; a larger lead angle could be provided with no other changes; a harder silicone could be used for the transition region with no other changes; the transition region could be overmolded with no other changes; the lead could have a combination of a tapered transition region and a greater lead angle with no other changes; or any other combination of the features referred to above.

Furthermore, it will be understood that the lead described above can be used in the standard Cochlear Surgical Technique, as well as other techniques including the Suprameatal Approach (SMA), as described in the paper entitled “The Suprameatal Approach in Cochlear Implant Surgery: Our Experience with 80 Patients”, published in ORL 2002; 64:403-405.

What is claimed is:

1-14. (canceled)

15. A cochlear implant electrode assembly for operably connecting to an implantable stimulator unit and having electrode contacts disposed at the distal end thereof for implantation into a cochlea of a patient, the electrode assembly comprising:
a helix region, adjacent the stimulator unit, containing wires connecting the stimulator unit to the electrode contacts, wherein the wires are wound in a helical arrangement;
an electrode array comprising an intra-cochlear region containing the electrode contacts and configured to be completely implanted in the cochlea, and a proximal region adjacent the intra-cochlear region and configured to be implanted extra cochlear; and
a transition region contiguous with and disposed between the helix and proximal regions, wherein the length of the transition region has a length of about 5 mm.

16. The electrode assembly of claim 15, wherein the length of the helix region is greater than about 1000% of the length of the transition region.

17. The electrode assembly of claim 15, wherein, the length of the helix region is between about 1000% and about 1300% of the length of the transition region.

18. The electrode assembly of claim 15, wherein the length of the helix region is about 63 mm.

19. The electrode assembly of claim 15, wherein the transition region tapers from the helix region to the proximal region.

20. The electrode assembly of claim 19, wherein the diameter of the transition region at the interface of the helix region and the transition region is about 1.2 mm.

21. The electrode assembly of claim 20, wherein the diameter of the transition region at the interface of the transition region and the proximal region is about 0.8 mm.

22. The electrode assembly of claim 15, wherein a lead angle between the transition region and the proximal region is greater than about 4 degrees.

23. The electrode assembly of claim 22, wherein the lead angle between the transition region and the proximal region is about 10 degrees.

24. The electrode assembly of claim 15, wherein the transition region tapers from the cross-sectional dimensions of the helix region to the cross-sectional dimensions of the proximal region.

25. The electrode assembly of claim 15, wherein the diameter of the transition region tapers from about 1.2 mm at the interface with the helix region down to about 0.8 mm at the interface with the proximal region.

26. The electrode assembly of claim 15, wherein the transition region is formed from a silicone having a Shore A hardness of 60.

27. A cochlear implant comprising:
an implantable stimulator unit; and
an electrode assembly operably connected to the stimulator unit and having electrode contacts disposed at the distal end thereof for implantation into a cochlea of a patient, the electrode assembly comprising:
a helix region, adjacent the stimulator unit, containing wires connecting the stimulator unit to the electrode contacts, wherein the wires are wound in a helical arrangement;
an electrode array comprising an intra-cochlear region containing the electrode contacts and configured to be completely implanted in the cochlea, and a proximal region adjacent the intra-cochlear region and configured to be implanted extra cochlear; and
a transition region contiguous with and disposed between the helix and proximal regions, wherein the length of the transition region has a length of about 5 mm.

28. The cochlear implant of claim 27, wherein the length of the helix region is greater than about 1000% of the length of the transition region.

29. The cochlear implant of claim 27, wherein, the length of the helix region is between about 1000% and about 1300% of the length of the transition region.

30. The cochlear implant of claim 27, wherein the length of the helix region is about 63 mm.

31. The cochlear implant of claim 27, wherein the transition region tapers from the helix region to the proximal region.

32. The cochlear implant of claim 31, wherein the diameter of the transition region at the interface of the helix region and the transition region is about 1.2 mm.

33. The cochlear implant of claim 32, wherein the diameter of the transition region at the interface of the transition region and the proximal region is about 0.8 mm.

34. The cochlear implant of claim 27, wherein a lead angle between the transition region and the proximal region is greater than about 4 degrees.

35. The cochlear implant of claim 34, wherein the lead angle between the transition region and the proximal region is about 10 degrees.

36. The cochlear implant of claim 27, wherein the transition region tapers from the cross-sectional dimensions of the helix region to the cross-sectional dimensions of the proximal region.

37. The cochlear implant of claim 27, wherein the transition region is formed from a silicone having a Shore A hardness of 60.

38. A cochlear implant electrode assembly for operably connecting to an implantable stimulator unit and having electrode contacts disposed at the distal end thereof for implantation into a cochlea of a patient, the electrode assembly comprising:
a helix region, adjacent the stimulator unit, containing wires connecting the stimulator unit to the electrode contacts, wherein the wires are wound in a helical arrangement; and
an electrode array contiguous with and adjacent to the helix region, the electrode array comprising an intra-cochlear region containing the electrode contacts and configured to be completely implanted in the cochlea, and a proximal region adjacent the intra-cochlear region and configured to be implanted extra cochlear.

39. The electrode assembly of claim 38, wherein a lead angle between the helix region and the proximal region is greater than about 4 degrees.
